(19) United States
(12) Patent Application Publication
(10) Pub. No.: US 2010/0104668 A1
(43) Pub. Date: Apr. 29, 2010

(54) NUTRITIONAL SUPPLEMENT FOR HONEYBEES

(76) Inventor: Mary Tereszkiewicz, Rainier, WA (US)

Correspondence Address: Marisha Tereszkiewicz, P.O. Box 633, Rainier, WA, 98576, US

(21) Appl. No.: 12/258,381

(22) Filed: Oct. 24, 2008

(51) Int. Cl. A61K 36/00; A61P 37/00; A61P 15/00; A61P 31/00

(52) U.S. Cl. 424/725

(57) ABSTRACT

A nutritional supplement for honeybees includes plant material chosen from a group comprised of plants known to provide nutrients to the honeybees, a beverage brewed from the plant material and a sugar base upon which the beverage is applied where, when the beverage has dried, the nutritional supplement is formed and can be distributed to supplement a normal honeybee feeding thereby providing a more balanced diet that at least in part helps to strengthen immune systems of the honeybees. The group includes plants known to, at least in part, stimulate a queen honeybee to lay eggs, stimulate hygienic behavior in a honeybee colony, provide a preventive maintenance against disease treat and reduce bacteria, fungus and viruses treat and reduce honeybee dysentery, reduce pests in a honeybee colony, such as tracheal and varroa mite and treat and reduce Deformed Wing Virus in a honeybee colony.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office, patent file or records, but otherwise reserves all copyright rights whatsoever.

### FIELD OF THE INVENTION

The present invention relates generally to honeybees (Apis Mellifera). More particularly, the invention relates to a nutritional supplement for honeybees that adds to a balanced diet, strengthens their immune system, stimulates the queen to lay eggs, and promotes hygienic behavior in the colony and a method for making such a supplement.

### BACKGROUND OF THE INVENTION

A third of our food is pollinated by the honeybee (Apis Mellifera) and there are no substitutes for the efficient job this creature does, yet the honeybee is in decline. The decline of the honeybee is known to those skilled in the art as colony collapse. No one knows the reason for colony collapse and no one has been able to stop it. Etymologists are looking in several areas to understand colony collapse. These areas include nutrition, genetically modified foods, pesticides, and diseases including viruses, bacteria, parasites and bee stresses such as transporting, climate change, etc. It is known that the honeybee does not have a strong immune system and seems ill equipped to fight off some of the stresses introduced to it.

A balanced diet promotes good health, and for a honeybee to maintain a balanced diet, it needs a variety of plants from which to forage. A honeybees' diet consists of nectar and pollen. These two ingredients provide the necessary proteins, amino acids, carbohydrates, sugar lipids (e.g., fatty acids and sterols), vitamins (e.g., B complex, thiamine, riboflavin, niacin, pyridoxine, pantothenate, folic acid, biotin, etc.), and minerals (e.g., salts) necessary for a healthy diet. A honeybee receives carbohydrates from nectars and protein from pollen. Protein is made of amino acids and not all plants contain all amino acids. When weeds were abundant, the honeybee was able to forage from a variety of perennials to maintain a balanced diet. But, with the introduction of genetically modified foods, hybrid seeds and weeds being killed by herbicides, the diet of the honeybee has been limited. Without a balanced diet, the honeybee's health suffers. For example, a decrease in any amino acid, such as, but not limited to, isoleucine, which is found in white clover and helps build muscle, may cause the survival of the colony to be jeopardized. Also, honeybee colonies that are being transported to work seasonal crops may not be exposed to the perennials they need to ensure colony health. For example, almond orchards and blueberry fields do not allow these weeds, which provide a balanced diet for the honeybee, to grow and consume the fertilizers meant for their crops. Therefore, the diet variety of the honeybee is reduced.

Currently known products that replicate the diet of the honeybee often use substitutes for the honeybees' natural diet. For example, protein patties, which are very popular and often fed inside the hive, are typically made of a mixture of canola, sunflower, and soy flour, sorghum and tricale, or brewers yeast and torula yeast. Each of these mixtures has advantages and disadvantages. Sugar is added in the form of granulated sucrose (i.e., table sugar), isomerized corn syrup (produced by multiple enzyme conversions of corn syrup and comprised largely of simple sugars such as dextrose, glucose and fructose), type-50 sugar syrup (containing equal amounts of dextrose and levulose), and cane or beet sugar. It is recommended that a minimum of 5% of naturally collected pollen be present. That means that typically only 5% of the food naturally collected by the honeybee is thought to be necessary to provide a healthy diet for the honeybee.

The standard for the nutritional needs of the honeybee was set by DeGroot in 1953. According to DeGroot, the amino acid Isoleucine, for example, should compose 4% of the honeybee's crude protein, and canola contains about 4.0% Isoleucine. However, since 1953, we have seen dichlorodiphenyltrichloroethane (DDT) come and go, we have experienced genetic engineering, and we are in the mists of developing genetically modified foods. Also, plants have evolved to a different state of being; few are seed producing and many are hybrids. With these new introductions, perhaps the values determined in 1953 no longer apply, and a supplement more closely resembling nectar and pollen would be beneficial to the honeybee diet. For example, one must question if the nutrition presently provided by a rose is equal to the nutrition that would have been provided by a rose in 1953. Yet, DeGroot's work is still referenced and used to calculate nutritional values for honeybee formulas.

Common honeybee nutritional products include; Megabee™ (a powder containing carbohydrates=47%, protein=40%, moisture=7%, fat=4% and ash=2%), Broodbuilder® (made with saccharomyces, which is a dairy yeast), Bee-Pro® and Feed-Bee® (both substitute pollen patties for bees). These are usually in powder form and must be mixed with syrup in order to be fed to the honeybees. All of these products have pros and cons regarding their nutritional values, usage, shelf life and palatability to bees.

Currently, no product on the market claims to boost the honeybee's immune system. However, Thymol, an essential oil from the thyme plant is sometimes added to the normal sucrose feed of honeybees to prevent tracheal mites, which are mites that live and multiply in the trachea tube of a honeybee and literally suffocate the honeybee. Menthol, an essential oil from the mint plant is also used as a substitute for thymol. Theses methods are currently known to those skilled in the art of beekeeping.

Broodbuilder®, Megabee™, and Bee-Pro®, (mentioned above) all claim to increase the number of eggs laid by the queen. They also claim to increase the amount of brood (i.e., young, eggs, larvae) in the colony. Nectar is nature's way of stimulating the queen to lay eggs. Once the workers begin bringing in nectar, the queen automatically begins increasing the brood in the colony. Sucrose, or white granulated cane sugar, mixed with water at a 1 to 1 ratio stimulates the queen to begin laying eggs. Beekeepers feed sucrose to the colony in the early spring and late fall to increase the number in the colony.

Currently there are no products that claim to promote hygienic behavior in honeybees. However, when the nectar flow begins naturally, bees become active, and their normal procedure is to begin cleaning the hive. Hygienic behavior is the behavior exhibited by honeybees to clean disease, bacteria, fungus, pests, dead bees and debris from the hive.

In view of the foregoing, there is a need for improved techniques for providing a nutritional supplement for honeybees that adds to a balanced diet, strengthens their immune system, stimulates the queen to lay eggs, and promotes hygienic behavior in the colony.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 is a flow chart illustrating an exemplary method for infusion to create a beverage for use as a nutritional supplement for honeybees, in accordance with an embodiment of the present invention;

FIG. 2 is a flow chart illustrating an exemplary method of decoction to create a beverage for use as a nutritional supplement for honeybees, in accordance with an embodiment of the present invention;

FIG. 3 is a flow chart illustrating an exemplary method of decoction to create a beverage for use as a nutritional supplement for honeybees, in accordance with an embodiment of the present invention; and

FIG. 4 is a flowchart illustrating an exemplary method for producing a nutritional supplement from a beverage made by infusion or decoction and feeding this nutritional supplement to a colony of honeybees, in accordance with an embodiment of the present invention.

Unless otherwise indicated illustrations in the figures are not necessarily drawn to scale.

### SUMMARY OF THE INVENTION

To achieve the forgoing and other objects and in accordance with the purpose of the invention, a nutritional supplement for honeybees and a method of making the nutritional supplement is presented.

In one embodiment, a method for providing a nutritional supplement to honeybees includes steps for creating a beverage for use in creating the nutritional supplement, steps for creating the nutritional supplement using the beverage and steps for feeding the honey bees whereby the honeybees are provided a more balanced diet that at least in part helps to strengthen immune systems of the honeybees. In various other embodiments, the steps for creating a beverage further include steps for choosing plant material that at least in part stimulates a queen honeybee to lay eggs, steps for choosing plant material that at least in part stimulates hygienic behavior in a honeybee colony, steps for choosing plant material that at least in part provides a preventive maintenance against disease in a honeybee colony, steps for choosing plant material that at least in part treats and reduces bacteria, fungus and viruses in a honeybee colony, steps for creating a beverage further comprise steps for choosing plant material that at least in part treats and reduces honeybee dysentery, steps for choosing plant material that at least in part reduces pests in a honeybee colony, such as tracheal and varroa mite and steps for choosing plant material that at least treats and reduces Deformed Wing Virus in a honeybee colony.

In another embodiment a nutritional supplement for honeybees includes plant material chosen from a group comprised of plants known to provide nutrients to the honeybees, a beverage brewed from the plant material and a sugar base upon which the beverage is applied where, when the beverage has dried, the nutritional supplement is formed and can be distributed to supplement a normal honeybee feeding thereby providing a more balanced diet that at least in part helps to strengthen immune systems of the honeybees. In various other embodiments the group is further comprised of plants known to at least in part stimulate a queen honeybee to lay eggs, the group is further comprised of plants known to at least in part stimulate hygienic behavior in a honeybee colony, the group is further comprised of plants known to at least in part provide a preventive maintenance against disease in a honeybee colony, the group is further comprised of plants known to at least in part treat and reduce bacteria, fungus and viruses in a honeybee colony, the group is further comprised of plants known to at least in part treat and reduce honeybee dysentery, the group is further comprised of plants known to at least in part reduce pests in a honeybee colony, such as tracheal and varroa mite and the group is further comprised of plants known to at least in part treat and reduce Deformed Wing Virus in a honeybee colony.

Other features, advantages, and object of the present invention will become more apparent and be more readily understood from the following detailed description, which should be read in conjunction with the accompanying drawings.

### DETAILED DESCRIPTION OF THE PREFERRED EMBODIMENTS

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

The present invention will now be described in detail with reference to embodiments thereof as illustrated in the accompanying drawings.

Preferred embodiments of the present invention provide a more balanced diet and help to strengthen the immune system of the honeybee (Apis Mellifera) through the methods of infusions and decoctions. Preferred embodiments also provide a natural way to stimulate the queen into egg laying and promote hygienic behavior in the colony. Infusions and decoctions have been around for a long time. Herbalists and Chinese medicines use them for medicinal reasons. The infusions and decoctions used in the present invention are made mostly from perennials, wild flowers and herbs. Many of these plants are thought of as weeds by most people.

When honeybees were abundant, their diet of nectar and pollen provided them with all the amino acids, proteins, carbohydrates, vitamins, minerals and fats that they needed. However, that was a half a century ago before genetically modified food, before hybrid and non-seed producing plants and before weeds were sprayed with herbicides. Because of these new introductions, the diet of the honeybee has been modified and limited and the immune system of the honeybee has been compromised. Preferred embodiments of the present invention are based upon the premise that these new genetically altered plants that are unable to reproduce themselves are vastly different from the plants 50 years ago that could reseed themselves. And, just as there is a difference in the makeup of the plant, there is likely a difference in the nectar and pollen produced by the plant. For example, without limitation, it is probable that the new altered hybrids and modified plants do not supply the honeybee with all of the nutrients in the correct amounts that they need for a balanced diet.

In a preferred embodiment, the infusions and decoctions are made from perennials that are typically thought of as weeds. However, at one time, these perennials were abundant upon the earth and the honeybee thrived on them. These perennials provide the honeybee with amino acids, carbohydrates, lipids, vitamins, fats, minerals, sugars, pollen, proteins, and other ingredients. By selecting perennials that have not been exposed to herbicides, pesticides or other sprayed chemicals and processing the perennials through the methods of infusion and/or decoction, active ingredients that were once available to the honeybee in abundance are extracted from the perennials. Using methods according to preferred embodiments, these perennials are steeped in boiling water for a short time to extract their active ingredients. This short time of exposure minimizes the loss of volatile elements from the perennials. The extracted elements in the water are then cooled and sprayed or dropped upon cane sucrose, which acts as a preservative. The supplement in this form can be stored and later added to the normal honeybee feedings or may be packaged and sold.

When supplements according to preferred embodiments of the present invention from infusions and decoctions are added to the honeybees' diet, the nutrients that are now limited or removed from plants via genetic manipulation are reintroduced into the diet. One must remember that we are humans giving our best guess to define a healthy honeybee's diet. We do not know for certain the nutritional needs of a honeybee; however we can compare the past to the present and recreate the diet the honeybee ingested when it was healthy and abundant. It should be noted that supplements according to embodiments of the present invention are nutritional supplements and do not replace pollen or nectar feeding at this time. These supplements are also not replacing the pollen patty or the white cane sugar feed; they are supplementing these feeds.

Including supplement according to preferred embodiments in the typical diet of a colony of honeybees provides many advantages including, but not limited to, the following advantages. Adding a greater variety of nutrients improves the overall general health of the colony because a more balanced diet promotes health. The honeybees' abilities to defend themselves against disease are enhanced because the supplement contains ingredients that strengthen the honeybees' bodily defense systems. These naturally occurring ingredients are nontoxic to the honeybee and human when ingested. Because the supplement replicates naturally occurring nectar flow, the supplement should stimulate the queen to begin laying more eggs. A strong colony with healthy bees has a greater survival rate. Increasing the colony population in the spring and autumn is recommended. The ‘nectar flow’ simulated by the supplement represents a truer nectar flow than the currently used white cane sugar. Nectar flow also stimulates hygienic behavior practices such as, but not limited to, the removal of dead bees, etc., and hygienic behavior is necessary to reduce disease in a colony.

Some preferred embodiments of the present invention produce a honeybee nutritional supplement by using the method of infusion. An infusion is a beverage made like tea where parts of the plant, usually including the flower right before it blooms and other parts such as, but not limited to, leaves and stems are immersed in boiling water and steeped in a nonmetal pot for approximately 10 minutes. This extracts the active ingredients of the plant parts, and the short period of time in which the plant matter is boiled minimizes the loss of volatile elements. The typical amounts used in this infusion are ½ to 1 ounce of plant matter to 1 pint of water. However, the amounts may vary to produce different strengths in alternate embodiments. Nutritional supplements according to preferred embodiments of the present invention may also be produces by decoctions. Decoctions are used to extract primarily the mineral salts and bitter parts of plants. Approximately ½ ounce of plant is boiled in 1 cup of water in a nonmetal pot in a decoction in preferred embodiments. Green parts of plants may be used in a decoction by being added to a nonmetallic pot and brought to a boil, allowing them to boil for approximately 3 to 5 minutes. Hard wood, roots, bark and seeds require this method to extract their nutrients and may take approximately 10 minutes of boiling.

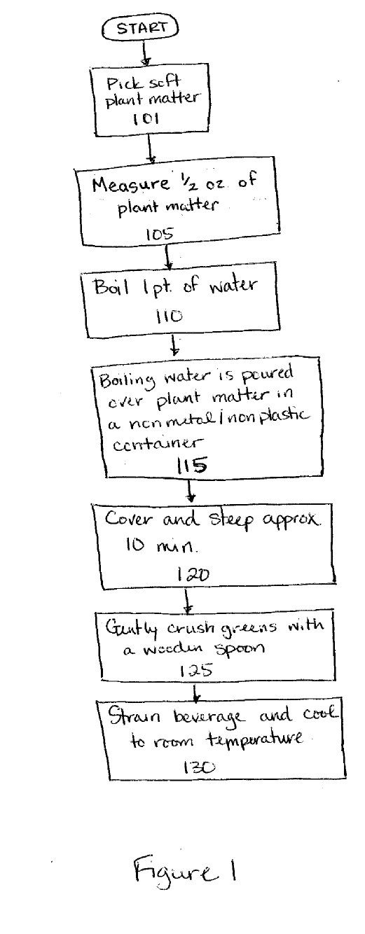

FIG. 1 is a flow chart illustrating an exemplary method of infusion to create a beverage for use as a nutritional supplement for honeybees, in accordance with an embodiment of the present invention. The following method is used to process soft plant matter. First in step 101 soft plant matter is picked just after the morning dew has dried and before the heat of the day. Flowers are picked just before they bloom, and leaves that are young are picked along with some newly formed stem pieces. The soft plant matter chosen should be free from chemicals and toxins. In step 105, ½ ounce of the plant matter (e.g., flowers, stems and leaves) is measured on a scale. The ratio of plant matter to water used in the present embodiment is ½ ounce of plant matter to one pint of water. However, in alternate embodiments the ratio of plant matter to water may be increased to increase the strength of the finished beverage or reduced to weaken the finished beverage. In the present embodiment, the one pint of water is boiled in step 110. In step 115 the plant matter is placed in a container that is not made of plastic or metal such as, but not limited to, a glass bowl, a nonmetal pot or ceramic container, and the boiling water is poured over the plant matter. In step 120 a tight fitting cover is placed over the top of the container to reduce evaporation, and the plant matter is left to steep for approximately 10 minutes. The steeping time may be increased or reduced to strengthen or weaken the finished beverage in alternate embodiments. If a tightly fitting cover is not used, some of the beverage may evaporate causing the end product to be weaker. In some cases where a weaker finished product is desired, a loose fitting cover may be used to enable some of the beverage to evaporate. Then, in step 125 of the present embodiment, the greens are gently crushed with a wooden spoon to release the oils from the plant matter. The beverage is strained in step 130, removing the plant matter, and cooled to room temperature. Those skilled in the art, in light of the present teachings, will readily recognize that some of the steps in the process previously described may be performed in various different orders. For example, without limitation, the water may be boiled before or while the plant matter is weighed.

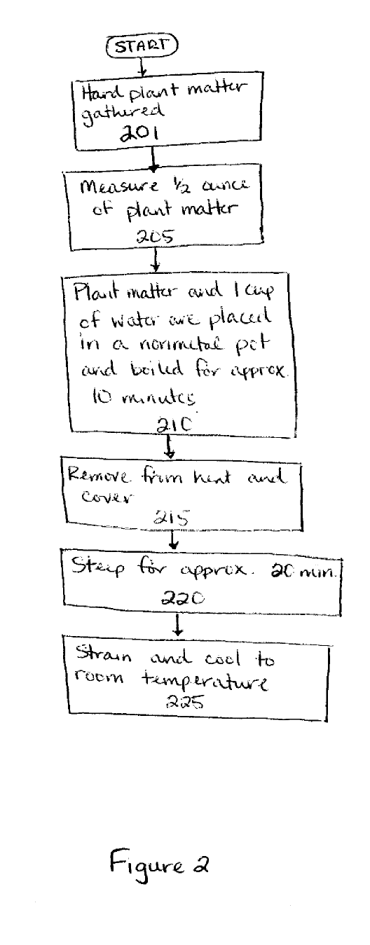

FIG. 2 is a flow chart illustrating an exemplary method of decoction to create a beverage for use as a nutritional supplement for honeybees, in accordance with an embodiment of the present invention. The following method is used to process hard plant matter. In step 201, hard plant matter such as, but not limited to, wood, root, bark, and seeds can be gathered when available. In step 205, ½ ounce of plant matter is measured on a scale. The ratio of plant matter to water used in the present embodiment is ½ ounce of plant matter to 1 cup of water. However, in alternate embodiments the ratio of plant matter to water may be increased to increase the strength of the finished beverage or reduced to weaken the finished beverage. In the present embodiment, in step 210, the plant matter is placed in a nonmetal pot with 1 cup of water and boiled for approximately 10 minutes. In alternate embodiments, the boiling time may be increased or reduced to strengthen or weaken the finished beverage. In step 215 of the present embodiment, the pot is removed from the heat, and a tight fitting cover is placed on the pot to reduce evaporation. If a tightly fitting cover is not used, some of the beverage may evaporate causing the end product to be weaker. In some cases where a weaker finished product is desired, a loose fitting cover may be used to enable some of the beverage to evaporate. In step 220, the plant matter is left to steep for approximately 20 minutes. The steeping time may be increased or reduced to strengthen or weaken the finished beverage in alternate embodiments. Then, in step 225 of the present embodiment, the beverage is strained, removing the plant matter, and cooled to room temperature. Those skilled in the art, in light of the present teachings, will readily recognize that some of the steps in the process previously described may be performed in various different orders.

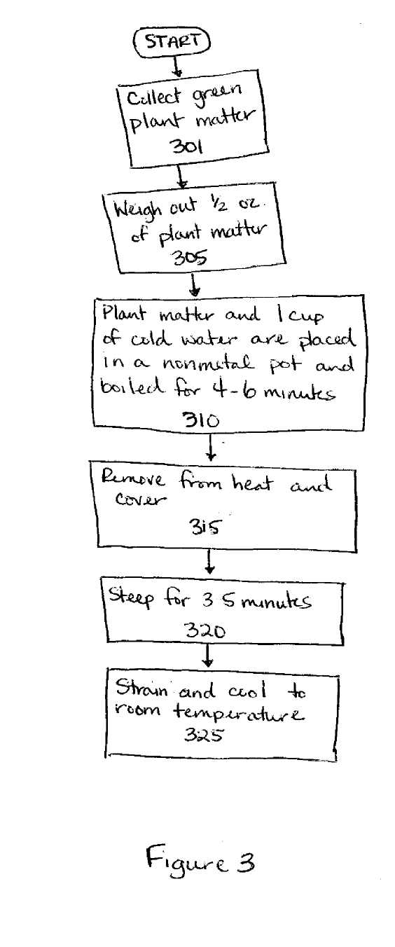

FIG. 3 is a flow chart illustrating an exemplary method of decoction to create a beverage for use as a nutritional supplement for honeybees, in accordance with an embodiment of the present invention. The following method is used to process green plant matter for decoctions. In step 301 green plant matter is collected as described by way of example in step 101 of FIG. 1. In step 305 ½ ounce of the green plant matter is weighed on a scale. The ratio of plant matter to water used in the present embodiment is ½ ounce of plant matter to 1 cup of cold water. However, in alternate embodiments the ratio of plant matter to water may be increased to increase the strength of the finished beverage or reduced to weaken the finished beverage. In the present embodiment, in step 310 the plant matter and 1 cup of cold water are placed in a nonmetal pot and boiled for 4 to 6 minutes. In alternate embodiments, the boiling time may be increased or reduced to strengthen or weaken the finished beverage. In step 315 of the present embodiment, the pot is removed from the heat, and a tight fitting cover is placed on the pot to reduce evaporation. If a tightly fitting cover is not used, some of the beverage may evaporate causing the end product to be weaker. In some cases where a weaker finished product is desired, a loose fitting cover may be used to enable some of the beverage to evaporate. In step 320, the plant matter is left to steep for approximately 3 to 5 minutes. The steeping time may be increased or reduced to strengthen or weaken the finished beverage in alternate embodiments. Then, in step 325 of the present embodiment, the beverage is strained, removing the plant matter, and cooled to room temperature. Those skilled in the art, in light of the present teachings, will readily recognize that some of the steps in the process previously described may be performed in various different orders.

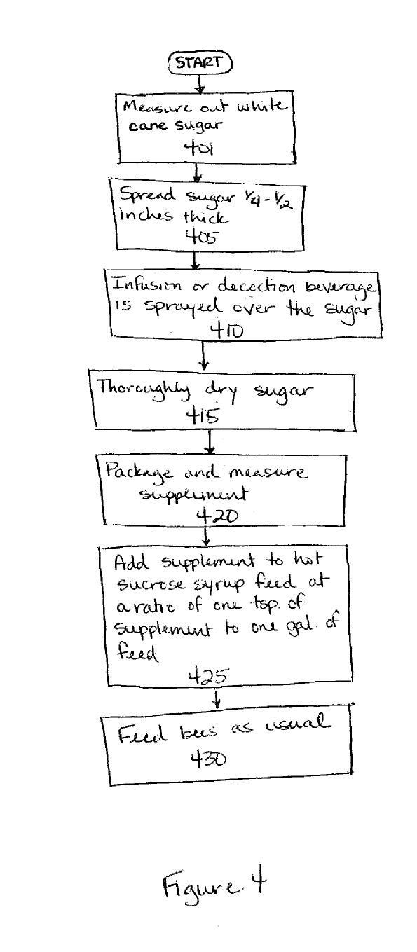

FIG. 4 is a flowchart illustrating an exemplary method for producing a nutritional supplement from a beverage made by infusion or decoction and feeding this nutritional supplement to a colony of honeybees, in accordance with an embodiment of the present invention. In step 401 the desired amount of white cane sugar is measured. The amount of white cane sugar used depends on the desired strength. Typically, one ounce of infusion may be sprayed on one cup of sugar or one ounce may be sprayed on two cups of sugar. The sugar is weighted, then packaged. Generally, the strength of the supplement will vary with the amount of sugar it is sprayed upon. In step 405 the sugar is spread approximately ¼ to ½ inches thick on a piece of plastic. In alternate embodiments to make the end product more cost-effective, the sugar may be spread thinner; however, the sugar should not be spread so thin as to lose effectiveness. In other alternate embodiments, the sugar may be spread thicker. In step 410, a beverage created from infusion or decoction for example, without limitation, a beverage made using one of the methods described by way of example in FIGS. 1 through 3 that is at room temperature is placed in a spray bottle and evenly dispersed over the sugar. Care should be taken to not over saturate the sugar with the beverage to cause the sugar to dissolve. In an alternate embodiment, using a dropper, the beverage may be applied to sugar cubes with 2 to 4 drops of the beverage being applied to each sugar cube where one sugar cube comprises 4 grams of sugar, again, taking care not to over saturate the sugar. The sugar cube should remain a cube and still resemble sugar; however, the sugar cube may be discolored or contain minute pieces of debris. In step 415 of the present embodiment the sugar is then allowed to thoroughly dry. This may take a day in open air. Once the sugar is dried, the supplement may be packaged and measured in step 420. For example, without limitation, in one version one pound of the supplement comprises the active ingredients from and infusion strength of ½ ounce-dandelion to one pint of water and is labeled as such on the package. In step 425, after sucrose syrup feed for a normal bee feeding is mixed and while the sucrose syrup feed is still hot, 1 teaspoon of the supplement is stirred into one gallon of feed. In alternate embodiments the ratio of supplement to feed may be increased or decreased. The supplement is not to be boiled in the feed. Then, in step 430, the bees are fed as usual. When adding the supplement made by infusion or decoction to the bee feed, it is possible and sometimes preferred to have several different products blended together. For example, without limitation, a beekeeper may add 1 tablespoon of a white clover product plus 2 teaspoons of a St. Johns wort product, plus 1 teaspoon of a lemon balm product along with the supplement.

In typical use of the preferred embodiment, the supplement is added to the normal sucrose (white cane sugar) fed to honeybees both in the spring and fall. Some of the perennials that may be used in the supplement such as, but not limited to dandelion and Echinacea have properties that help the body of the honeybee defend itself from invading bacteria, fungus or viruses. The beekeeper can add this supplemental product into his routine syrup feed to be ingested by the honeybee. Upon ingestion, the honeybee will either eat the food, thereby consuming the nutrients or place the food in its honey stomach and deliver the food to a place in the hive for storage. If the bee places the food in storage, the ingredients from the supplement will be preserved in the honey until consumed at a later date. For example, without limitation, if one wishes to increase the antibacterial defenses of larvae, by theory, a supplement made from the infusion of a plant known to have antibacterial properties may be added to the feed, and these properties would be stored in the cell and fed to the larvae, enhancing the larvae's immune system.

Honeybees consume proteins in the form of pollen. These proteins are made of amino acids that are consumed by the nurse bees who produce Royal Jelly from their hypopharyngeal gland and pack newly laid eggs in the Royal Jelly. Royal Jelly is fed to the queen and larvae. If the nurse bees are deficient in a particular amino acid, because the amino acid is not available to the foraging bee, the deficiency is passed to the larvae and the health of the colony is affected. By using the supplement according to preferred embodiments of the present invention, a beekeeper can expose the colony to a greater variety of nutrients.

It is known to those skilled in the art that the flow of nectar stimulates the hive to behave in a hygienic manner; dead bees are removed and the hive cleaned of fungus and bacteria. Currently, white cane sugar is used as nectar however, it is lacking in many ingredients that compose true natural nectar. The supplements made from the infusions and decoctions replicate true nectar closer than plain sucrose (white cane sugar).

As previously mentioned, nature's normal nectar flow in the spring stimulates the queen to begin laying eggs as it is an indicator that food is now becoming available and time to rebuild the colony from the winter's loss. Beekeepers typically mimic this natural process by feeding sugar syrup to the bees in the spring and the autumn, to tease the queen into building up the colony.

White cane sugar (i.e., sucrose) sometimes with a drop or two of menthol essential oils to prevent trachea mites is generally used by beekeepers to stimulate the queen to lay eggs in the spring and the autumn. The stronger the colony in the spring, the greater the honey produced and the pollination potential, and having a strong colony entering the winter increases the survival rate of the colony as large colonies provide more warmth during the winter. Supplements according to embodiments of the present invention may be added to sucrose (granulated white cane sugar) syrup. The queen is already stimulated by the syrup; however, the supplement, which more resembles a natural nectar flow, stimulates the queen's egg production further. Based upon a trial conducted in the autumn, it was found that sucrose syrup with a supplement according to an embodiment of the present invention added increased egg production and therefore increased the size of the colonies. The colonies built up nicely. It was also found that the honeybees consumed the sucrose including the supplement at least three times faster than the sucrose alone. Furthermore, an infusion of a plant that prevents trachea mites may be added into the supplement so that menthol essential oils do not need to be used.

Since nectar flow and hygienic behavior go hand in hand, supplements according to embodiments of the present invention, which closely replicate natural nectar flow, should promote hygienic behavior in the colony. Also as the queen increases egg production, there are more in-hive bees available to clean the hive and nurse the young bees.

Various different types of plants may be used to create supplements according to preferred embodiments of the present invention. Pollen provides the protein for the honeybee. Amino acids, which make up proteins, are different in number and types in various plants. There has been research conducted regarding which plants are visited most frequently by honeybees. With this information, the plants most visited by honeybees can be identified along with the amino acids in these plants, and supplements can be created to closely mimic the amino acids of the most visited plants. Giving credit to the intelligence of the honeybee; it knows what it needs, the job of beekeepers is to observe the honeybee and provide them the nutrients, in the correct quantities, so they will be healthy and prolific. Furthermore, different types of plants with healing and disease prevention properties may be used in the infusions or decoctions to create supplements to treat or prevent specific diseases or conditions. For example, without limitation, adding the infusions or decoctions of herbs can help treat various Apis Mellifera diseases. In one such example an infusion of mint may be used to help relieve the honeybee of tracheal mites. In another non-limiting example, an infusion of dandelion may be used as an antibacterial agent against Nosema, honeybee dysentery. The potentials are practically unlimited.

Supplements according to preferred embodiments of the present invention may be used to create a line of products that are mixed together based upon amino acids, fats, minerals, vitamins and carbohydrates all taken from the infusions and decoctions of non-altered, nontoxic plant matter, preferably weeds. These products may then be used as a base on which to create various other products. For example, without limitation, the extract from the infusion of white clover may be increased in some products. By increasing the white clover infusion, the amino acid isoleucine, which helps in building muscle tissue, is increased. Therefore, the base product may be enhanced to promote specific areas of honeybee health. In another non-limiting example, when a particular virus breaks out that effects the nervous system of the honeybee, the base product may be enhanced to better defend the honeybee at a very early larvae stage. The use of this product should result in healthy immune systems and increased survival rates of the honeybee.

Supplements according to preferred embodiments of the present invention may also be used as a new way to medicate a colony of honey bees, inside the hive and inside the cell, right down to the larvae. In contrast, traditional medication methods include the dusting of bees with powders, feeding patties made from ingredients that honeybees do not typically eat or having the beekeeper mask and glove up to apply formic acid, which is the treatment for Varroa mite. In the application of formic acid extra care must be taken so the formic acid does not contaminate the honey. By using infusions and decoctions with antibacterial and antifungal properties, the honeybee is better prepared to defend itself against these invaders in the future. This method of medicating via infusions and decoctions would change the way honeybees are medicated. There would be a focus on prevention and a greater understanding of the interconnections between all life: plant, insect and human.

Supplements according to preferred embodiments of the present invention are all natural, comprising no chemicals. The supplements can be consumed by humans without any injurious side effects. This is important since the sucrose feed given to the bees is used to produce honey. What is fed to the bees eventually makes its way back to the human. Some imported honey has contained chemicals or drugs such as, but not limited to, chloramphenicol, and penicillin. The plant matter selected to make preferred embodiments are free of chemicals and pesticides so that these chemicals do not make their way into the diets of humans. Furthermore, these supplements are easily added to bee feedings, and the bees seem to like the supplements, eating them quickly. The supplements also have no side effects to the bees, unlike some currently used chemicals added to fight diseases in the hive, which actually may kill bees. Amounts and strengths of the supplements can be easily manipulated, and they seem to be a natural way to boost the Apis Mellifera's immune system.

Preferred embodiments may also increase the survival rate of larvae, by adding ingredients that will be stored in the bee bread (food made by honeybees) and later fed to the newly hatched larvae. Currently known thymol products are extracted from thyme and are used against varroa mites. These products are placed inside the hive, smell terrible and are usually applied three times approximately 10 days apart. Increasing the thymol in bee bread via supplements, ather than applying thymol products to the hive, is less work for the beekeeper.

The advantage of introducing nontoxic, naturally occurring plant nutrients into the colony at a later time via in-hive syrup feeding, are notable. It is a method that provides a more balanced diet for the honeybee when plants are no longer in season. Some beekeepers extract all of the honey from the hive in autumn to sell or consume and feed the bees white cane sugar. The beekeepers may or may not supplement this white cane sugar with pollen patties. The extracted honey, the stored winter food for the bees, comprises more nutrition than the plain sucrose fed to them to replenish their winter supply. Supplements according to embodiments of the present invention can be added to the sucrose feed, adding nutrients to their feed and providing the bees with a more nutritional winter diet. This would also be beneficial in climate change when erratic weather patterns prevent the honeybee from foraging to obtain nutrients.

Supplements according to embodiments of the present invention provide a more balanced nutritional diet, which leads to a healthy colony. These naturally occurring properties found in some plant matter aid in boosting the immune system of the honeybee by providing amino acids, vitamins and minerals which add to the general health of the bee. Also, a healthy bee is better able to defend itself from invading bacteria, viruses and fungus.

Practically every beekeeper is looking for a fast, safe and cheap way to keep their bees healthy. Supplements according to embodiments of the present invention can satisfy this need of the beekeepers. Furthermore, climate changes have shown us that we are all interconnected and need to respect life. Because of this, among other reasons, many people are going “green”. Supplements according to embodiments of the present invention provide an all-natural “green” product that also aids in the well-being of another species.

In alternate embodiments, there may be variations in when the soft or hard plant matter is picked or gathered. It is possible to create the same product if the plant matter is gathered at various times of day and year. The strengths of various embodiments may also vary with the amount of plant matter used. Product strength is dependent upon factors such as, but not limited to, the amount, type and parts of plant matter used. The strength of each product is different and almost impossible to accurately measure. Therefore, to label packaging, it is preferred to list the plant matter and method used to make the product, for example, without limitation, “made from ¼ ounce of red clover infused in 1 pint of water for approximately 5 minutes”.

The types of plants used may also vary in alternate embodiments to focus on advantages of the particular embodiment such as, but not limited to, immune system building or amino acids to enhance brain activity in the honeybee. The combination of plants may also vary in alternate embodiments. Plant matter of choice is obtained from perennials. However, if particular ingredients are not available in perennials at that time yet are present in annuals or if perennials cannot be found, annuals may be substituted for perennials to generally ensure that the resulting product contained the desired ingredients. Hardiness, being non-hybrids and the ability to germinate seeds that can reproduce the plant make perennials the better choice. However, it is conceivable that embodiments of the present invention can be made with only annuals or some annuals, semi-annuals, bi-annuals and perennials, resulting in a less effective product.

Alternate embodiments may use various different methods for extracting the ingredients from the plant matter such as, but not limited to juicing, cold pressing and distillation and fluid extractions. In embodiments using fluid extractions, the fluid extract is made by techniques that use multiple solvent extractions, resulting in a very concentrated product.

Alternate embodiments of the present invention may also use tinctures. Tinctures include storing herbs, perennials and wildflowers in alcohol, and may be necessary to eliminate storage problems. Tinctures afford the option of gathering the plant material when the plants are in season, and then storing the plant matter to be used at a later date. Storing the products in alcohol would also create a tincture.

Having fully described at least one embodiment of the present invention, other equivalent or alternative methods of providing nutritional supplements for honeybees according to the present invention will be apparent to those skilled in the art. The invention has been described above by way of illustration, and the specific embodiments disclosed are not intended to limit the invention to the particular forms disclosed. The invention is thus to cover all modifications, equivalents, and alternatives falling within the spirit and scope of the following claims.

What is claimed is:

1. A method for providing a nutritional supplement to honeybees, the method comprising:
steps for creating a beverage for use in creating the nutritional supplement;
steps for creating the nutritional supplement using said beverage; and
steps for feeding the honey bees whereby the honeybees are provided a more balanced diet that at least in part helps to strengthen immune systems of the honeybees.

2. The method as recited in claim 1, wherein said steps for creating a beverage further comprise steps for choosing plant material that at least in part stimulates a queen honeybee to lay eggs.

3 The method as recited in claim 1, wherein said steps for creating a beverage further comprise steps for choosing plant material that at least in part stimulates hygienic behavior in a honeybee colony.

4. The method as recited in claim 1, wherein said steps for creating a beverage further comprise steps for choosing plant material that at least in part provides a preventive maintenance against disease in a honeybee colony.

5. The method as recited in claim 1, wherein said steps for creating a beverage further comprise steps for choosing plant material that at least in part treats and reduces bacteria, fungus and viruses in a honeybee colony.

6. The method as recited in claim 1, wherein said steps for creating a beverage further comprise steps for choosing plant material that at least in part treats and reduces honeybee dysentery.

7. The method as recited in claim 1, wherein said steps for creating a beverage further comprise steps for choosing plant material that at least in part reduces pests in a honeybee colony, such as tracheal and varroa mite.

8. The method as recited in claim 1, wherein said steps for creating a beverage further comprise steps for choosing plant material that at least treats and reduces Deformed Wing Virus in a honeybee colony.

9. A nutritional supplement for honeybees comprising:
plant material chosen from a group comprised of plants known to provide nutrients to the honeybees;
a beverage brewed from said plant material;
a sugar base upon which said beverage is applied where, when said beverage has dried, the nutritional supplement is formed and can be distributed to supplement a normal honeybee feeding thereby providing a more balanced diet that at least in part helps to strengthen immune systems of the honeybees.

10. The nutritional supplement for honeybees as recited in claim 9, wherein said group is further comprised of plants known to at least in part stimulate a queen honeybee to lay eggs.

11. The nutritional supplement for honeybees as recited in claim 9, wherein said group is further comprised of plants known to at least in part stimulate hygienic behavior in a honeybee colony.

12. The nutritional supplement for honeybees as recited in claim 9, wherein said group is further comprised of plants known to at least in part provide a preventive maintenance against disease in a honeybee colony.

13. The nutritional supplement for honeybees as recited in claim 9, wherein said group is further comprised of plants known to at least in part treat and reduce bacteria, fungus and viruses in a honeybee colony.

14. The nutritional supplement for honeybees as recited in claim 9, wherein said group is further comprised of plants known to at least in part treat and reduce honeybee dysentery.

15. The nutritional supplement for honeybees as recited in claim 9, wherein said group is further comprised of plants known to at least in part reduce pests in a honeybee colony, such as tracheal and varroa mite.

16. The nutritional supplement for honeybees as recited in claim 9, wherein said group is further comprised of plants known to at least in part treat and reduce Deformed Wing Virus in a honeybee colony.
